(19) United States
(12) Patent Application Publication
(10) Pub. No.: US 2019/0350462 A1
(43) Pub. Date: Nov. 21, 2019

(54) METHODS AND DEVICES FOR MEASURING BODY TEMPERATURE IN A REDUCED TIME

(71) Applicant: Verily Life Sciences LLC, South San Francisco, CA (US)

(72) Inventors: William Biederman, San Francisco, CA (US); Sean Frick, San Francisco, CA (US); Roxana Heitz, Mountain View, CA (US)

(21) Appl. No.: 16/410,961

(22) Filed: May 13, 2019

Related U.S. Application Data
(60) Provisional application No. 62/671,784, filed on May 15, 2018.

(51) Int. Cl. A61B 5/01; A61B 5/00; A61F 7/02

(52) U.S. Cl. CPC A61B 5/01; A61B 5/6843; A61B 5/7225; A61F 2007/0086; A61B 5/6833; A61F 7/02; A61B 5/0008; A61B 5/7275

(57) ABSTRACT

Various methods and devices for reducing the time for a body-mountable temperature monitoring device to provide an accurate body temperature are disclosed. Methods are used to accurately predict the measured temperature once the monitoring device reaches thermal equilibrium. For example, in one embodiment, a method for measuring body temperature of a subject includes obtaining, by a temperature monitor attached to a body of the subject, a plurality of body temperature measurements at a plurality of times, determining parameters of a model used for predicting temperature based on the plurality of measurements, predicting a body temperature of the subject based on the model, and outputting the predicted body temperature as a current temperature reading.

### RELATED APPLICATIONS

The present application claims the benefit of the filing date of U.S. Provisional Patent Application No. 62/671,784, filed May 15, 2018, the disclosure of which is hereby incorporated by reference in its entirety.

### TECHNICAL FIELD

The present disclosure relates generally to physiological monitoring and, in particular, to wearable devices and associated methods for monitoring a patient's temperature. For example, a wearable temperature monitoring device may include one or more heating elements to provide for accurate body temperature measurements in a short amount of time.

### BACKGROUND

A long-term goal of health-related wearable electronics is to gather a patient's health data in real time to provide remote, continuous monitoring of human health. A temperature monitoring device typically requires an initial warm up period that can last at least a few minutes, thereby delaying the ability to accurately monitor temperature. This time delay in measurement can be caused by the heat capacity of a sensing element of the temperature monitoring device and the fact that applying the temperature monitoring device to the patient tissues or skin draws down the temperature of the tissues or skin in the immediate region of the temperature monitoring device.

For example, body-mountable temperature monitoring devices typically require several minutes (e.g., 5-10 minutes) to reach a steady-state temperature once applied to the body due to their thermal mass. Therefore, the temperature reading from these devices can be highly inaccurate during that time. This causes an inconvenient delay for users who want to measure body temperature quickly.

### SUMMARY

Various methods and devices for reducing the time for a body-mountable temperature monitoring device to provide an accurate body temperature are disclosed. Methods are used to accurately predict the measured temperature once the monitoring device reaches thermal equilibrium. A body-mountable device may include one or more separate heating elements to accelerate the device reaching thermal equilibrium. In some embodiments, the devices and methods use both temperature prediction and a heating element to provide an accurate body temperature in a shorter amount of time. For example, the heating element can be turned on and then the body temperature can be predicted, or the heating element can be turned on for a short amount of time and turned off, and then the temperature can be predicted.

For example, in one embodiment, a method for measuring body temperature of a subject includes: obtaining, by a temperature monitor attached to a body of the subject, a plurality of body temperature measurements at a plurality of times for the subject; determining, by the temperature monitor, parameters of a model used for predicting temperature, based on the plurality of measurements; predicting, by the temperature monitor, a body temperature of the subject based on the model; and outputting, by the temperature monitor, the predicted body temperature as a current temperature reading.

In some embodiments, the model comprises an exponential model for temperature, and wherein determining the parameters of the model comprises determining values of a plurality of parameters in the exponential model. In some embodiments, the model comprises:

t(time)=(Tₑ−T₀)(1−e^{-αt})+T₀,

where t=time, T₀=an initial temperature, Tₑ=a final temperature, and α=a thermal time constant, and wherein determining the parameters of the model comprises determining values of α and Tₑ. In some embodiments, predicting a body temperature of the subject based on the model comprises: determining a value of a of the model; and inputting a time into the model.

In some aspects, the method further comprises: continuing to obtain a plurality of body temperature measurements at a plurality of times after the predicted body temperature is outputted; and updating the predicted body temperature based on the continued obtainment of the plurality of body temperature measurements at a plurality of times. In some embodiments, the plurality of times comprises predetermined intervals. In other aspects, the method further comprises activating a heating element coupled to the temperature monitor. In some embodiments, the heating element is activated when the temperature monitor detects that the temperature monitor is attached to the body of the subject. In some embodiments, activating the heating element comprises activating the heating element for a predetermined amount of time; and wherein obtaining the plurality of body temperature measurements comprises obtaining the plurality of body temperature measurements over a period of time that is greater than the predetermined amount of time.

According to another embodiment of the present disclosure, a temperature monitoring device for measuring body temperature of a subject includes: a thermal sensor configured to sense a body temperature and to obtain a plurality of body temperature measurements at a plurality of times for the subject; and a microcontroller configured to: receive the plurality of measurements from the thermal sensor, determine parameters of a model used for predicting body temperature based on the plurality of measurements, predict a body temperature of the subject based on the model, and output the predicted body temperature as a current temperature reading.

In some embodiments, the model comprises:

t(time)=(Tₑ−T₀)(1−e^{-αt})+T₀,

where t=time, T₀=an initial temperature, Tₑ=a final temperature, and α=a thermal time constant, and wherein determining the parameters of the model comprises determining values of α and Tₑ. In some embodiments, the microcontroller is further configured to: continue to obtain a plurality of body temperature measurement at a plurality of times after the predicted body temperature is outputted; and update the predicted body temperature based on the continued obtainment of the plurality of body temperature measurements at a plurality of times.

In some aspects, the device includes a thermal contact configured to contact the body of the subject and a heating element adjacent to the thermal contact, wherein the microcontroller is configured to activate the heating element. In still other embodiments, the microcontroller is further configured to: detect that the temperature monitoring device is attached to the body of the subject, and activate the heating element upon the detection. In other aspects, the device further includes an adhesive patch incorporating the heating element.

According to another embodiment of the present disclosure, a temperature monitoring device comprises: a thermal contact configured to contact a body of the subject; a heating element adjacent to the thermal contact and configured to heat the thermal contact; a thermal sensor configured to sense a temperature of the thermal contact and to obtain a plurality of body temperature measurements at a plurality of times for a time greater than a predetermined amount of time; and a microcontroller configured to: activate the heating element for the predetermined amount of time, receive the plurality of measurements from the thermal sensor, determine parameters used for predicting body temperature based on the plurality of measurements, predict a body temperature of the subject based on the parameters, and output the predicted body temperature as a current temperature reading.

In some embodiments, the parameters are part of a model and the model comprises:

t(time)=(Tₑ−T₀)(1−e^{-αt})+T₀,

where t=time, T₀=an initial temperature, Tₑ=a final temperature, and α=a thermal time constant, and wherein determining the parameters of the model comprises determining values of α and Tₑ. In some embodiments, the microcontroller is further configured to: continue to obtain a plurality of body temperature measurement at a plurality of times after the predicted body temperature is outputted; and update the predicted body temperature based on the continued obtainment of the plurality of body temperature measurements at a plurality of times.

Additional aspects, features, and advantages of the present disclosure will become apparent from the following detailed description

### BRIEF DESCRIPTION OF THE DRAWINGS

The following figures are included to illustrate certain aspects of the present invention, and should not be viewed as an exclusive embodiment. The subject matter disclosed is capable of considerable modification, alteration, and equivalents in form and function, as will occur to those of ordinary skill in the art and having the benefit of this disclosure.

FIG. 1 illustrates a system for wirelessly measuring the body temperature of a subject using a body-mountable temperature monitoring device according to one or more embodiments of the present disclosure;

FIG. 2 schematically illustrates a body-mountable temperature monitoring device that may be used according to one or more embodiments of the present disclosure;

FIG. 3 illustrates an exploded cross-sectional view of an exemplary body-mountable temperature monitoring device that may be used according to one or more embodiments of the present disclosure;

FIG. 4 illustrates a graph that shows the variation in detected body temperature over time according to one or more embodiments;

FIG. 5 depicts a first method for measuring body temperature in a reduced time according to one or more embodiments;

FIG. 6 depicts a second method for measuring body temperature in a reduced time according to one or more embodiments;

FIG. 7 depicts a third method for measuring body temperature in a reduced time according to one or more embodiments; and

FIG. 8 depicts a fourth method for measuring body temperature in a reduced time according to one or more embodiments.

### DETAILED DESCRIPTION

For the purposes of promoting an understanding of the principles of the present disclosure, reference will now be made to the embodiments illustrated in the drawings, and specific language will be used to describe the same. It is nevertheless understood that no limitation to the scope of the disclosure is intended. Any alterations and further modifications to the described devices, systems, and methods, and any further application of the principles of the present disclosure are fully contemplated and included within the present disclosure as would normally occur to one skilled in the art to which the disclosure relates. In particular, it is fully contemplated that the features, components, and/or steps described with respect to one embodiment may be combined with the features, components, and/or steps described with respect to other embodiments of the present disclosure. For the sake of brevity, however, the numerous iterations of these combinations will not be described separately.

The methods described herein provide a quick response in monitoring the body temperature of a subject using a body-mountable temperature monitoring device or a “temperature monitor.” When the temperature monitor is first attached to the subject's body (or re-attached to the body after detachment), there exists a significant thermal unbalance between the subject's body and the temperature monitor. Until the temperature monitor reaches thermal equilibrium with the subject's body, the temperature readout does not accurately reflect the body temperature of the subject. Once thermal equilibrium is reached (i.e., the temperature reported by the temperature monitor has reached a constant level), the body temperature of the subject can be determined and accurately reported.

In one embodiment, the methods sample multiple body temperatures for a certain amount of time and predict what the final body temperature will be based on the sampled body temperatures. In another embodiment, the methods activate a heating element of the temperature monitor to speed up or accelerate the time it takes for the temperature monitor to reach thermal equilibrium. In some embodiments, the methods include both prediction of the final body temperature and use of the heating element.

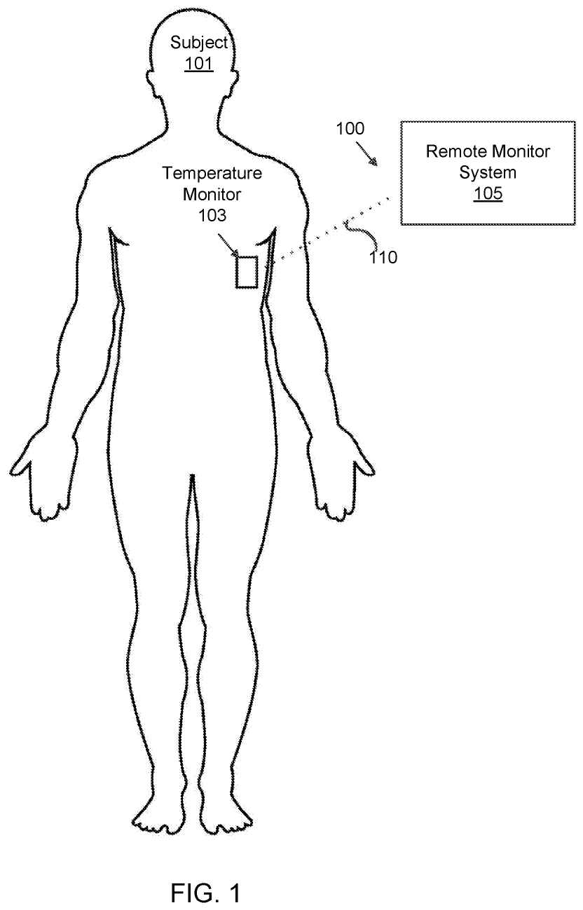

FIG. 1 illustrates an exemplary system 100 for wirelessly sensing the body temperature of a subject 101. As shown, the system 100 includes a temperature monitor 103 and a remote monitoring system 105. The temperature monitor 103 can send temperature information (e.g., a visual readout of the core body temperature of the subject 101 and other related information) to the remote monitoring system 105, where the temperature information can be shown on a display. Alternatively, the temperature monitor 103 may include a display where the information is provided.

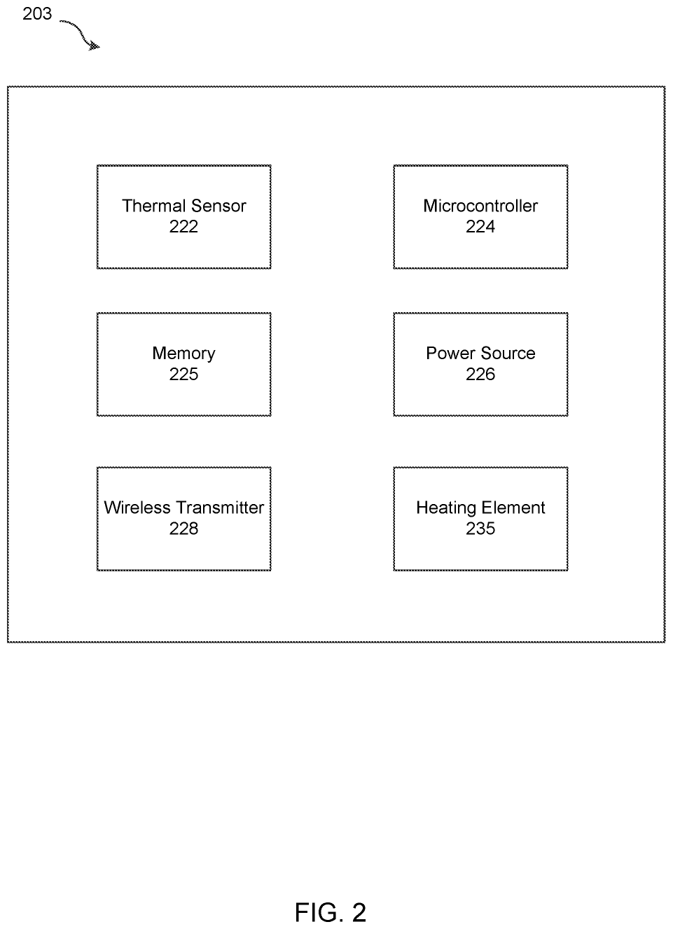

In one or more embodiments, the temperature monitor 103 may be worn or carried by the subject 101. In various embodiments, the temperature monitor 103 is removably attached to a portion of the subject's body or the subject's skin. For example, the temperature monitor 103 can be worn on different locations of the subject's body, such as the forehead, torso, neck, arm, leg, or other suitable locations. In exemplary embodiments, the temperature monitor 103 is implemented as a wearable patch that is attachable to the skin of the subject 101, as will be further explained below and in FIG. 2.

The remote monitoring system 105 communicates with the temperature monitor 103. The remote monitoring system 105 can receive signals from the temperature monitor 103 via the wireless communication link 110. The wireless communication link 110 can be established as short range wireless communication, such as radio frequency identification (RFID) communication, near field communication (NFC), Bluetooth communication, or Wi-Fi communication.

In some embodiments, the remote monitoring system 105 is operable to present the data transmitted from the temperature monitor 103 on a display. For example, the remote monitoring system 105 may include a display screen and operate to present the transmitted data on the screen in a visible format. The remote monitoring system 105 can also output the data in an audible format, and/or provide an alert in visible and/or audible ways.

The remote monitoring system 105 can be used by a guardian and/or a healthcare practitioner to monitor the measurement of the temperature monitor 103. Examples of the guardian include a parent of the subject 101, a family member of the subject 101, a caregiver of the subject 101, a primary physician of the subject 101, and any other interested parties. The healthcare practitioner is a person who provides healthcare service to the subject 101. Examples of healthcare practitioners include primary care providers (e.g., doctors, nurse practitioners, and physician assistants), nursing care providers (e.g., nurses), specialty care providers (e.g., professionals in various specialties), and health professionals that provide preventive, curative, promotional and rehabilitative health care services. The healthcare practitioner can be an institution, company, business, and/or entity. In other embodiments, the temperature monitor 103 can be operated by the subject 101 him or herself.

The remote monitoring system 105 can be of various types. In various embodiments, the remote monitoring system 105 is a computing device dedicated to particular temperature monitors 103. In other embodiments, other consumer level computing devices can be used for the remote monitoring system 105. Such computing devices can include a mobile computing device, such as a smartphone, a tablet computer, and a personal digital assistant (PDA). The remote monitoring system 105 can include a desktop computer, a laptop computer, and/or any other suitable devices operable to send and receive signals, store and retrieve data, and/or execute modules.

FIG. 2 illustrates a block diagram of a temperature monitor 203. As shown, the temperature monitor 203 includes a sensor or plurality of sensors 222 (e.g., a thermal sensor), a microcontroller 224, a memory 225, a power source 226, a wireless transmitter 228, and optionally a heating element 235. In general, the sensor 222 operates to detect the body temperature of the subject 101, and may include one sensor or a plurality of sensors. The microcontroller 224 operates to process signals received from the sensor 222. Data processed by the microcontroller 224 can be stored in the memory 225. The memory 225 provides the microcontroller 224 with non-transitory, computer-readable storage to facilitate execution of computer instructions by the microcontroller 224. The power source 226 supplies power to the components of the temperature monitor 203. The wireless transmitter 228 operates to send signals obtained by the temperature monitor 203 to the remote monitoring system 105 via the wireless communication link 110. The heating element 235, when present, operates to heat a thermal contact that provides the body temperature of the subject 101 to the sensor 222.

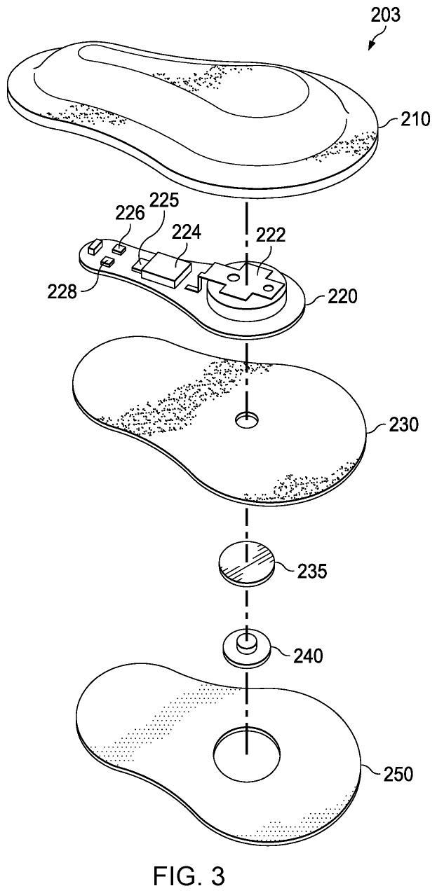

FIG. 3 illustrates an exemplary temperature monitor 203. As shown, temperature monitor 203 includes an upper elastomeric housing 210, a printed circuit board 220, a bottom elastomeric housing 230, a thermal contact 240, and an adhesive patch 250. Consistent with one embodiment, a heating element 235 is included adjacent thermal contact 240. The heating element 235 is optional and need not be included in all embodiments. The temperature monitor 203 may be utilized for monitoring of infants and children, elderly patients, and hospitalized patients.

The temperature monitor 203 includes an enclosure that includes the upper elastomeric housing 210 and the bottom elastomeric housing 230. The upper and lower elastomer housings 210 and 230 can be any biocompatible housing or casing configured to shield components of the temperature monitor 203. The enclosure can be constructed of various materials, including plastics and/or rubbers that provide durability and moisture resistance to the temperature monitor 203.

The temperature monitor 203 further includes a printed circuit board 220 that can communicate information to the remote monitoring system 105. The printed circuit board 220 typically includes a sensor 222, a microcontroller 224, a memory 225, a power source 226, and a wireless transmitter 228. The sensor 222 can be a multitude of sensors, including a thermal sensor that senses temperature, such as a thermistor, a resistor temperature detector, a thermocouple, or any other miniature temperature responsive sensor, and other types of sensors, such as an ambient temperature sensor that detects ambient air temperature of the environment of the subject 101 and/or an accelerometer that detects movement/activity and body orientation. In some embodiments, the temperature monitor 203 includes a contact temperature sensor (referred to in some embodiments as a primary temperature sensor) that measures temperature based on contact, and one or more other temperature sensors (referred to in some embodiments as secondary temperature sensors), which may be contact temperature sensors or non-contact temperature sensors (e.g., an infrared sensor), or a combination of these. The secondary temperature sensor(s) give a measure of ambient temperature in the vicinity of the temperature monitor 203.

In one embodiment, the sensor 222 includes a thermal sensor. In other embodiments, the sensor 222 includes a thermal sensor and one or more other sensors configured to detect and measure environmental effects that may impact the detected body temperature. For example, the sensor 222 may include one or more sensors that detect contextual data such as a microphone or other acoustic sensor to detect sounds and other environmental noises, relative and global positioning sensors to determine the location of the subject 101, torque and rotation acceleration sensors to determine orientation of the subject 101 in space, and sensors configured to detect pollen, humidity, ozone, and other environmental conditions. Contextual data as used herein means data relating to the environment, surroundings, location and condition of the subject 101, including, but not limited to, air quality, audio sound quality, ambient temperature, ambient light, global positioning, humidity, altitude, barometric pressure, and the like. Contextual data may also include further abstractions or derivations regarding the condition and status of the subject 101.

The microcontroller 224 is configured to estimate body temperature of the subject 101 based, at least in part, on the temperature measurements of the thermal sensor. Additionally, the microcontroller 224 can use inputs from other sensors in addition to the temperature measurements from the thermal sensor to compensate and estimate body temperature. Advantageously, the microcontroller 224 can utilize thermal sensor readings in combination with other sensor readings to adjust the detected body temperature.

In certain embodiments, the microcontroller 224 receives data from multiple sensors on the temperature monitor 203 to adjust temperature readings to account for environmental and other conditions. For example, in hot, humid conditions, it is difficult for the body to dissipate heat because of the moisture in the air and small temperature gradient between the skin and the ambient air (e.g., as measured by one or more contact temperature sensors and one or more secondary temperature sensors, respectively), resulting in an elevated body temperature. On the other hand, in frigid or windy conditions, a great deal of metabolic work is necessary to produce body heat and prevent it from leaving the body, resulting in a drop in body temperature. In another example, physical activity (e.g., exercise) can affect body temperature. The response of the body to physical activity is an increase in metabolic rate, resulting in increased heat production within the body and elevated body temperature.

In various embodiments, the microcontroller 224 takes into account differences between temperatures detected by a thermal sensor and an ambient temperature sensor, how fast temperature readings are changing, and the duration of changing temperature readings to output a more accurate estimate of core body temperature of the subject 101. If the subject 101 is determined to be in a hot, humid environment, the body temperature of the subject 101 that is output can be adjusted downwards to account for the effect of the weather.

In some embodiments, the microcontroller 224 provides an activity classification to apply corresponding temperature adjustment parameters to the subject 101. For example, the microcontroller 224 can determine the activity level of the subject 101 using an accelerometer and humidity sensors. If the activity classification of the subject 101 is high, the body temperature of the subject 101 that is output can be adjusted downwards to account for the effect of physical activity.

In several embodiments, a confidence qualifier is attached to the data, which is based on the temperature differences and the activity classification. The confidence qualifier indicates the probability or likelihood that the actual body temperature falls within the outputted body temperature. For example, if large temperature differences and a high activity classification are determined, the confidence qualifier is a lower value than if small temperature differences and a low activity classification is determined.

In some embodiments, the microcontroller 224 processes the readings from primary and secondary sensors to calculate the heat transfer between body and environment and estimate core body temperature using the following steps. When primary and secondary temperature sensor measurements are within a few tenths of degrees Celsius of each other and are not changing, a high confidence is assigned that the determined temperature represents core temperature. When a measured temperature from a secondary sensor drops/increases, followed by drop/increase, respectively, in measured temperature from a primary sensor, the microcontroller 224 may determine that the temperature measured by the primary sensor is affected by changes in the vicinity of the secondary sensor. In this scenario, final core temperature estimates may be determined by fitting exponentials and can then calculate core temperature assuming a thermal gradient, as above. The more data points available for the fit, the higher the confidence in the predicted result. Likewise, when a measured temperature from the primary sensor drops/increases, followed by drop/increase, respectively, in a measured temperature from a secondary sensor, in a method it may be determined that the secondary sensor measurement is affected by change in conditions in the vicinity of the primary sensor (e.g., skin temperature) and the underlying core temperature estimate may take these changes into account.

In additional embodiments, the data received from various sensors is subject to adaptive filtering, such as assigning different weights to different temperature measurements from different sensors at different times. In some embodiments, data is averaged and higher confidence data (e.g., data with a higher confidence qualifier) is provided greater weight in the determination of body temperature.

In various embodiments, the microcontroller 224 is a small computer or other circuitry that retrieves and executes software from memory. In exemplary embodiments, the microcontroller 224 is configured to perform any necessary computations to deliver an equilibrium temperature of the temperature monitor 203. For example, the microcontroller 224 can be programmed and/or otherwise configured to run the algorithms necessary to predict body temperature and other related data. In some embodiments, the microcontroller 224 is implemented as an application-specific integrated circuit (ASIC), a field-programmable gate array (FPGA), application-specific standard part (ASSP), a system-on-chip (SoC), a digital signal processor (DSP), or a general-purpose processor. In various embodiments, a power source 226, such as a battery, is provided. Some embodiments of the temperature monitor 203 also include a storage device or memory 225 for storing instructions for microcontroller 224, such as random access memory (RAM) devices including dynamic RAM (DRAM), synchronous DRAM (SDRAM), solid state memory devices, and/or a variety of other memory devices known in the art In one more embodiments, a wireless transmitter 228 is provided.

A thermal contact 240 is also provided in the temperature monitor 203. The thermal contact 240 has a surface adapted for thermal coupling with the skin of the subject 101. The thermal contact 240 is generally made of any conductive material. In some embodiments, the thermal contact 240 is a gold-plated brass disc that facilitates thermal coupling with the skin of the subject 101. The thermal sensor senses the temperature of the thermal contact 240.

The heating element 235 can be disposed on the printed circuit board 220 or embedded in the adhesive patch 250. The heating element may be implemented as a resistive heater, a microheater, or a positive temperature coefficient (PTC) thermistor. In some embodiments, the heating element 235 is implemented as a wire loop embedded in the printed circuit board 220. This could be heated from an external source, for example, by passing current through the wire. Instead of a resistive wire loop, one or more resistors can be implemented on the printed circuit board 220 to achieve the desired heating function. In one embodiment, the heating element 235 is thermally coupled to the thermal contact 240. In other embodiments, the heating element 235 can be incorporated directly into the adhesive patch 250 and thermally coupled to the thermal contact 240, as described below. In various embodiments, the heating element 235 can be on the printed circuit board or on, around, and/or under the adhesive patch 250. For example, a heater wire can be warmed up when a voltage is applied from a contact to the printed circuit board 220.

The heating element 235 is typically deactivated or turned off after a predetermined amount of time (e.g., before thermal equilibrium is reached or before the warm up period ends) at a predetermined temperature level. The heating element 235 can accelerate the rate at which the thermal contact 240 warms up, reducing the total time for the thermal contact 240 to reach thermal equilibrium with the body of the subject 101. In other words, use of the heating element 235 reduces the warm up period. In one or more embodiments, the heating element 235 is activated for a short amount of time (e.g., less than a minute) to provide a quick burst of heat, and then turned off to allow the temperature monitor 203 to settle.

An adhesive patch 250 allows the temperature monitor 203 to be removably attached to the skin of the subject 101. The adhesive patch 250 can be constructed of various materials, including plastics, or natural or synthetic fibers. The materials are chosen to provide durability and breathability to the temperature monitor 203. In some embodiments, the adhesive patch 250 includes a biocompatible adhesive. A film or paper can be pulled away from the adhesive patch 250 prior to applying the temperature monitor 203 to the skin of the subject 101. The adhesive patch 250 can include a hydrogel, which can provide skin-adhesion properties. In addition or alternatively, the adhesive patch 250 can include a pressure-sensitive adhesive. The adhesive patch 250 generally includes a cutout or opening for the thermal contact 240.

The adhesive patch 250 can further function as a thermal conduit between the thermal sensor/thermal contact 240 and the skin of the subject 101. For example, the adhesive patch 250 can include biocompatible thermally and electrically conductive materials to improve thermal contact reliability. Suitable materials include silver nanowires, carbon nanotubes, silver, gold, platinum, activated carbon powders (ACP) and major conducting polymer materials, namely, polyaniline, polypyrrole, polythiophene and derivatives of polythiophene, as well as composites of these materials with carbon nanotubes. The electrical conductivity of these materials may improve wireless communication and range performance by creating a larger, solid ground plane, which may improve antenna radiation efficiency. In some embodiments, non-biocompatible materials (e.g., copper) can be used as long as the non-biocompatible materials are sealed and/or are not in contact with the body of the subject 101.

As explained above, temperature monitoring devices typically require several minutes to warm up and reach thermal equilibrium, which results in highly inaccurate temperature readings until the devices reach a stabilized temperature. Generally, when the temperature monitor 203 is attached to the skin of the subject 101, the skin of the subject 101 will transfer heat energy to the thermal contact 240, or vice versa, depending on ambient conditions. Eventually, the thermal contact 240 will reach a state of thermal equilibrium, in which the temperature of the thermal contact 240 of the monitor 203 is relatively stable and substantially matches the body temperature of the subject 101. Using conventional techniques, it is only after the thermal contact 240 reaches thermal equilibrium that an accurate body temperature can be sensed by the thermal sensor and provided to a user.

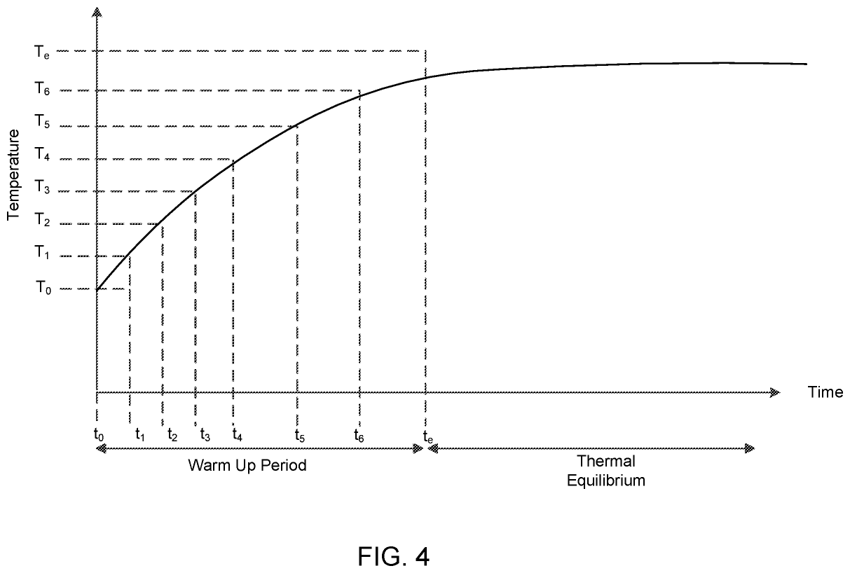

FIG. 4 illustrates the variation in temperature over time as the temperature monitor 203 detects the body temperature of the subject 101. Initially, the thermal contact 240 is at ambient or room temperature (To). In this example, ambient temperature is lower than body temperature (e.g., 65° Fahrenheit (F) to 75° F.), but the methods also apply if ambient temperature is higher than body temperature. After attaching the temperature monitor 203 to the skin of the subject 101, heat flow from the subject's body will raise the temperature of the thermal contact 240, so that the thermal sensor will report rising temperatures (T₁-T₆). Ultimately, the temperature of the thermal contact 240 will reach an equilibrium temperature (Tₑ) that is sensed by the thermal sensor.

As described herein, the thermal sensor is not ready to immediately detect the body temperature of the subject 101 when the temperature monitor 203 and associated thermal contact 240 is first attached to a body portion (e.g., skin or tissues) of the subject 101. Such measurement time delay can occur for various reasons. Such reasons can include the construction of the temperature monitor 203 including the location of the thermal contact 240 within the temperature monitor 203, the heat capacity of the thermal contact 240, a difference in temperature between the thermal contact 240 and the subject's body, and an improper attachment of the temperature monitor 203 and/or thermal contact 240 on the subject's body. Because of such various reasons, when the temperature monitor 203 is first engaged with the subject's body, the temperature detected by the thermal sensor can be unstable or fluctuate during the initial warm up period (e.g., t₁-t₆ as illustrated in FIG. 3) until the thermal contact 240 becomes in thermal equilibrium with the body portion on which the temperature monitor 203 is attached. As the thermal contact 240 and the engaged body portion become in thermal equilibrium, the temperature detected by the temperature monitor 203 is stabilized, and thus can be used as an accurate measurement of the subject's body temperature.

As such, the temperature monitor 203 typically requires some time until it can reliably measure the body temperature of the subject 101. It is preferable, however, to take body temperature measurements as quickly as possible in many situations.

Advantageously, the methods and devices described herein reduce the amount of time it takes to obtain an accurate reading of the body temperature of the subject 101 in two ways. First, the methods predict the final stabilized body temperature by taking a plurality of temperature readings, and then curve fit these data points to determine where the curve will settle. Second, the methods accelerate the time the thermal contact 240 takes to reach thermal equilibrium so that a final stabilized body temperature is reached faster.

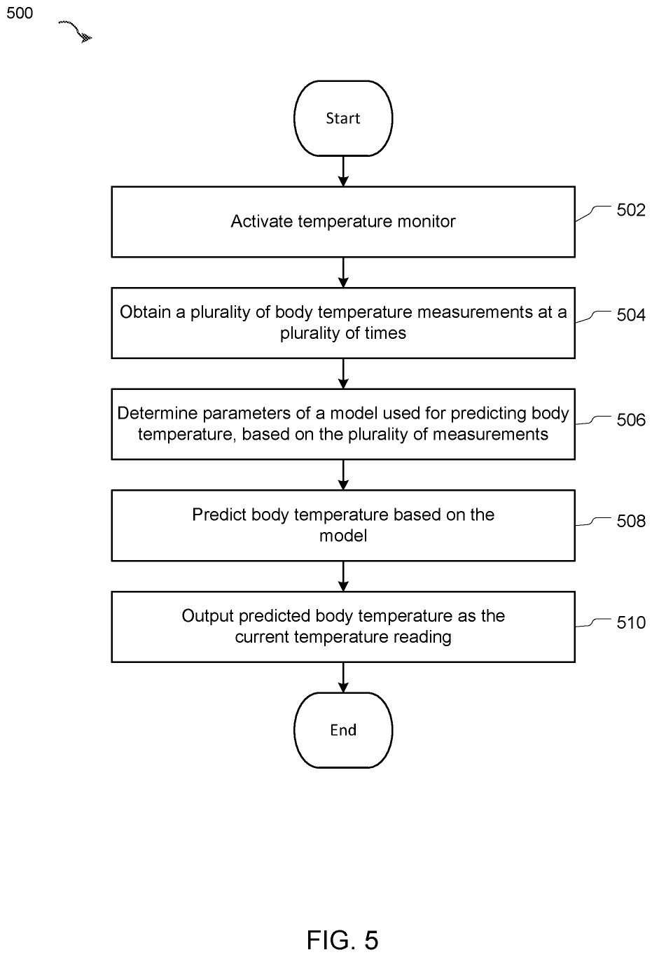

FIG. 5 is a flowchart illustrating an exemplary method 500 for measuring a body temperature of a subject in a reduced time. The method 500 begins when the temperature monitor 203 is arranged in place on a predetermined location of the body of the subject 101 and activated at step 502. At step 504, the temperature monitor 203 obtains a plurality of body temperature measurements at a plurality of times. For example, the temperature monitor 203 can sample 10 temperature readings at 10 different times. The plurality of times may be at predetermined intervals (e.g., every second, every 5 seconds, etc.), and the intervals need not be the same amount of time. For example, referring back to FIG. 4, the thermal sensor can provide a temperature T₁ at time t₁, a temperature T₂ at time t₂, a temperature T₃ at time t₃, a temperature T₄ at time t₄, a temperature T₅ at time t₅, and a temperature T₆ at time t₆ to the microcontroller 224. The time between t₁ and t₂ does not need to be the same amount of time as the time between t₂ and t₃.

At step 506, the temperature monitor 203 determines parameters of a model used for predicting body temperature, based on the plurality of measurements. For example, referring back to FIG. 4, the microcontroller 224 plots data points temperature T₁ at time t₁, temperature T₂ at time t₂, temperature T₃ at time t₃, temperature T₄ at time t₄, temperature T₅ at time t₅, and temperature T₆ at time t₆, on a graph and connect the data points with a curve. In several embodiments, the microcontroller 224 determines the curve equation from the plurality of data points. There are a variety of potential models to use to predict temperature. In one model, the rate of change of temperature is assumed to be proportional to the difference between the temperature of the monitor 203 and the temperature of the body, yielding an exponential model. For example, in one embodiment, the curve equation for the curve in FIG. 4 is:

t(time)=(Tₑ−T₀)(1−e^{-αt})+T₀,

where t=time, T₀ is the initial temperature, Tₑ=the final temperature and α=a thermal time constant. Tₑ and k are unknown quantities to be determined by curve fitting or solving for these values. By substituting the data points for temperature and time into the equation, the values for the unknown quantities can be obtained and one or more future temperatures predicted. Other types of curves (e.g., a polynomial model, a combination of exponential models, or a combination of a polynomial model and an exponential model) may be used. For example, experimental measurement data in different scenarios may be stored either on the body-wearable device or remote device used to predict temperature and used to predict temperature. In other embodiments, “learned” parameters from the device and the uses can be stored. For example, after the first few fits, the model can refine itself.

At step 508, the temperature monitor 203 predicts the body temperature of the subject based on the model. For example, the microcontroller 224 uses the plotted curve to determine at what temperature the curve will settle or stabilize. In another example, the microcontroller 224 determines the approximate time the temperature of the temperature monitor 203 will stabilize (e.g., 5-10 minutes) and then inputs the time into the curve equation to yield the stabilized body temperature.

At step 510, the temperature monitor 203 outputs the predicted body temperature as the current temperature reading. This final projected value of the body temperature can be transmitted as the current temperature instead of the actual current temperature detected by the temperature monitor 203. In some embodiments, the method 500 can be completed in about a minute.

In several embodiments, the algorithm on the microcontroller 224 can continue running during the warm up period, and the projected final value of the body temperature can continue to be updated and refined as more data points are used for the exponential fit. In this way, higher fit accuracy can be achieved.

Accordingly, if it is found that the temperature detected by the temperature monitor 203 is not stabilized yet, temperature monitor 203 can be configured to predict the body temperature of the subject 101 for quicker body temperature determination, even during the warm up period, thereby reducing or eliminating delay in providing feedback to a user, particularly as compared to waiting until thermal equilibrium is achieved before communicating body temperature.

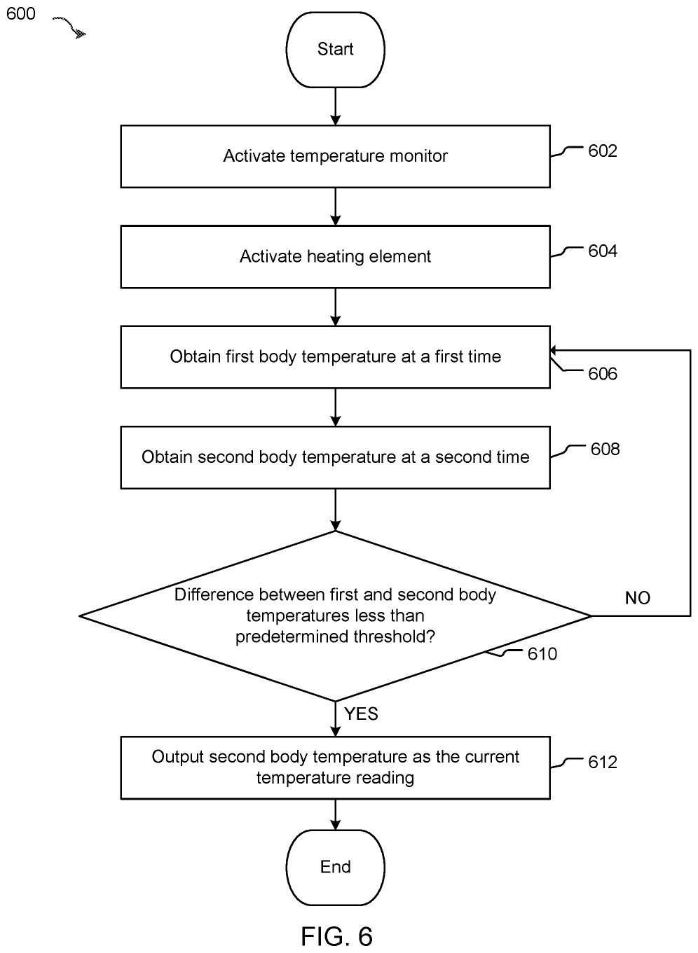

FIG. 6 is a flowchart illustrating another exemplary method 600 for measuring a body temperature of a subject in a reduced time. The method 600 begins when the temperature monitor 203 is arranged in place on a predetermined location of the body of the subject 101 and activated at step 602.

At step 604, a heating element 235 of the temperature monitor 203 is activated. The heating element 235 can be activated when the temperature monitor 203 detects that the thermal contact 240 is placed on the body. For example, the temperature monitor 203 can activate the heating element 235 upon detection of a rise in temperature, or when a button is pressed, or using capacitive sensing as examples. Moreover, the temperature monitor 203 can include a second temperature sensor for sensing ambient temperature and the system can activate the heating element 235 when the thermal contact temperature deviates from the sensed ambient temperature.

At step 606, the temperature monitor 203 obtains a first body temperature at a first time. At step 608, the temperature monitor 203 obtains a second body temperature at a second time. For example, the first time in step 606 is set as a time (i.e., to in FIG. 4) at which, or shortly after, the temperature monitor 203 is activated. The second time in step 608 is set as a predetermined time (i.e., t₁ in FIG. 4) after the temperature monitor 203 is activated. In other embodiments, the first and second times can be different points in time at different intervals after the temperature monitor 203 is activated. For example, in FIG. 4, the first and second times can be any combination selected from t₀, t₁, t₂, t₃, t₄, t₅, and t₆ to the extent that the first time is before the second time.

At step 610, the temperature monitor 203 calculates the difference between the first and second body temperatures to determine if the difference is less than a predetermined threshold. In general, during the initial warm up period, the temperature monitor 203 obtains at least two body temperatures at different times, determines a rate of body temperature increase, and compares the rate with a predetermined threshold to indicate that the body temperature has been sufficiently stabilized to provide a reliable reading. The threshold is set close to zero to represent that the body temperature is so stabilized over time as to provide a reliable reading on the body temperature.

If the temperature difference between the two body temperatures does not meet the predetermined threshold, the temperature monitor 203 returns to steps 606 and 608. If the temperature difference between the two body temperatures does meet the predetermined threshold, the method 600 continues to step 612, where the temperature monitor 203 outputs the second body temperature as the current temperature reading.

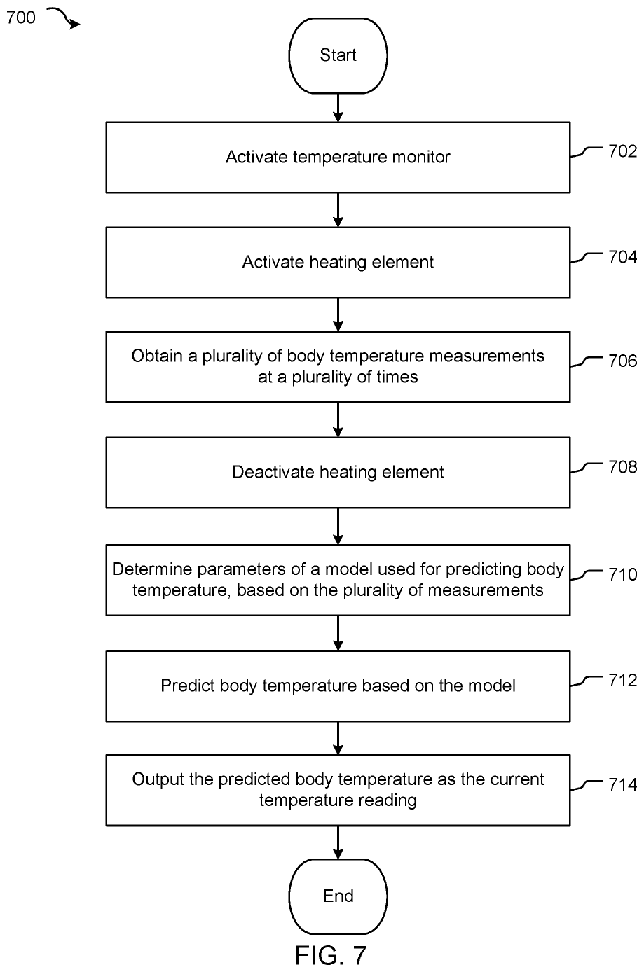

FIG. 7 is a flowchart illustrating another exemplary method 700 for measuring a body temperature of a subject in a reduced time. The method 700 employs a model and use of a heating element.

The method 700 begins when the temperature monitor 203 is arranged in place on a predetermined location of the body of the subject 101 and activated at step 702. At step 704, the temperature monitor 203 activates the heating element 235, for example, upon detecting that the temperature monitor 203 is placed on the body of the subject 101, similar to step 604 in method 600.

At step 706, the temperature monitor 203 obtains a plurality of body temperature measurements at a plurality of times. Step 706 is similar to step 504 in method 500. At step 708, the temperature monitor 203 deactivates the heating element 235. The heating element 235 is activated while the temperature monitor 203 obtains a plurality of body temperatures at a plurality of times for determining parameters of the model.

At step 710, the temperature monitor 203 determines parameters of a model used for predicting body temperature, based on the plurality of measurement. Step 708 is similar to steps 506 in method 500.

At step 712, the temperature monitor 203 predicts the body temperature based on the model. At step 714, the temperature monitor 203 outputs the predicted body temperature as the current temperature reading. Steps 712 and 714 are similar to steps 508 and 510 in method 500.

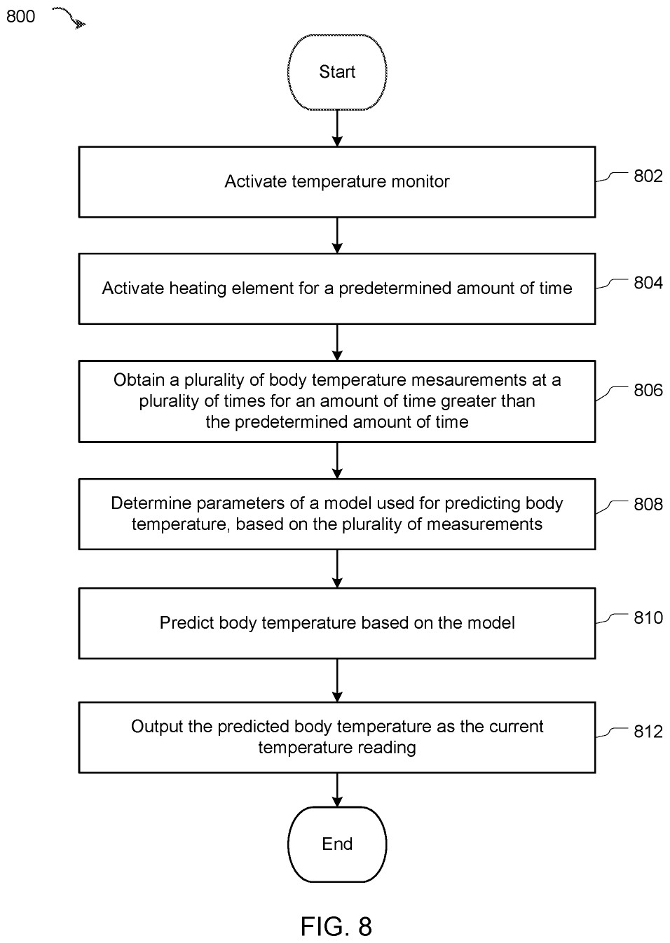

FIG. 8 is a flowchart illustrating another exemplary method 800 for measuring a body temperature of a subject in a reduced time. The method 800 also employs a model and use of a heating element.

The method 800 begins when the temperature monitor 203 is arranged in place on a predetermined location of the body of the subject 101 and activated at step 802. At step 804, the temperature monitor 203 activates the heating element 235 for a predetermined amount of time (e.g., for an amount of time shorter than the warm up period) upon detecting that the temperature monitor 203 is placed on the body of the subject 101.

At step 806, the temperature monitor 203 obtains a plurality of body temperature measurements at a plurality of times for an amount of time greater than the predetermined amount of time. That is, the heating element 235 is deactivated before the temperature monitor 203 obtains a sufficient number of body temperature measurements at different times.

At step 808, the temperature monitor 203 determines parameters of a model used for predicting body temperature, based on the plurality of measurements. At step 810, the temperature monitor predicts the body temperature based on the model. At step 812, the temperature monitor 203 outputs the predicted body temperature as the current temperature reading. Steps 806-812 are similar to steps 504-510 in method 500.

According to one or more embodiments, a method for measuring body temperature of a subject is provided. Embodiments of the method may generally include obtaining, by a temperature monitor attached to a body of the subject, a plurality of body temperature measurements at a plurality of times for the subject; determining, by the temperature monitor, parameters of a model used for predicting temperature, based on the plurality of measurements; predicting, by the temperature monitor, a body temperature of the subject based on the model; and outputting, by the temperature monitor, the predicted body temperature as a current temperature reading.

In some embodiments, the model includes an exponential model for temperature, and determining the parameters of the model includes determining values of a plurality of parameters in the exponential model. In various embodiments, the model includes:

t(time)=(Tₑ−T₀)(1−e^{-αt})+T₀,

where t=time, T₀=an initial temperature, Tₑ=a final temperature, and α=a thermal time constant, and determining the parameters of the model includes determining values of α and Tₑ. In several embodiments, predicting a body temperature of the subject based on the model includes determining a value of a of the model; and inputting a time into the model.

In one or more embodiments, the method further includes continuing to obtain a plurality of body temperature measurements at a plurality of times after the predicted body temperature is outputted; and updating the predicted body temperature based on the continued obtainment of the plurality of body temperature measurements at a plurality of times.

In certain embodiments, the plurality of times includes predetermined intervals.

According to one or more embodiments, a temperature monitoring device for measuring body temperature of a subject is provided. The temperature monitoring device generally includes a thermal sensor configured to sense a body temperature and to obtain a plurality of body temperature measurements at a plurality of times for the subject; and a microcontroller configured to receive the plurality of measurements from the thermal sensor; determine parameters of a model used for predicting body temperature based on the plurality of measurements, predict a body temperature of the subject based on the model, and output the predicted body temperature as a current temperature reading.

In some embodiments, the model includes:

t(time)=(Tₑ−T₀)(1−e^{-αt})+T₀,

where t=time, T₀=an initial temperature, Tₑ=a final temperature, and α=a thermal time constant, and determining the parameters of the model includes determining values of α and Tₑ.

In several embodiments, the microcontroller is further configured to continue to obtain a plurality of body temperature measurement at a plurality of times after the predicted body temperature is outputted; and update the predicted body temperature based on the continued obtainment of the plurality of body temperature measurements at a plurality of times.

According to one or more embodiments, another method of measuring body temperature of a subject is provided. The method generally includes activating a heating element of a temperature monitor attached to a body of the subject; obtaining, by the temperature monitor, a first body temperature of the subject at a first time; obtaining, by the temperature monitor, a second body temperature of the subject at a second time; calculating, by the temperature monitor, a difference between the first body temperature and the second body temperature; determining, by the temperature monitor, that the difference is less than a predetermined threshold; and outputting, by the temperature monitor, the second body temperature as a current temperature reading.

In certain embodiments, the heating element is activated when the temperature monitor detects that the temperature monitor is attached to the body of the subject. In some embodiments, the heating element is embedded in an adhesive patch of the temperature monitor.

According to one or more embodiments, another temperature monitoring device is provided. The temperature monitoring device generally includes a thermal contact configured to contact a body of the subject; a heating element adjacent to the thermal contact and configured to heat the thermal contact; a thermal sensor configured to sense a temperature of the thermal contact and to obtain a first body temperature of the subject at a first time and a second body temperature of the subject at a second time; and a microcontroller configured to activate the heating element, receive the first and second body temperatures from the thermal sensor, calculate a difference between the first and second body temperatures, determine that the difference is less than a predetermined threshold, and output the second body temperature as a current temperature reading.

In several embodiments, the microcontroller is further configured to detect that the temperature monitoring device is attached to the body of the subject, and activate the heating element upon the detection.

In some embodiments, the temperature monitoring device further includes an adhesive patch incorporating the heating element.

According to one or more embodiments, yet another method of measuring body temperature of a subject is provided. The method generally includes activating a heating element of a temperature monitor attached to a body of the subject; obtaining, by the temperature monitor, a plurality of body temperature measurements at a plurality of times for the subject; deactivating the heating element after obtaining the plurality of body temperature measurements at a plurality of times; determining, by the temperature monitor, parameters of a model used for predicting body temperature, based on the plurality of measurements; predicting, by the temperature monitor, a body temperature based on the model; and outputting, by the temperature monitor, the predicted body temperature as a current temperature reading.

In certain embodiments, the model includes an exponential model for temperature, and determining the parameters of the model includes determining values of a plurality of parameters in the exponential model. In one or more embodiments, the model includes:

t(time)=(Tₑ−T₀)(1−e^{-αt})+T₀,

where t=time, T₀=an initial temperature, Tₑ=a final temperature, and α=a thermal time constant, and determining the parameters of the model includes determining values of α and Tₑ.

In several embodiments, predicting a body temperature of the subject based on the model includes determining a value of a of the model; and inputting a time into the model.

In some embodiments, the method further includes continuing to obtain a plurality of body temperature measurements at a plurality of times after the predicted body temperature is outputted; and updating the predicted body temperature based on the continued obtainment of the plurality of body temperature measurements at a plurality of times. In one embodiment, the plurality of times includes predetermined intervals.

In certain embodiments, the heating element is activated when the temperature monitor detects that the temperature monitor is attached to the body of the subject. In other embodiments, the heating element is embedded in an adhesive patch of the temperature monitor.

According to one or more embodiments, another temperature monitoring device is provided. Embodiments of the temperature monitoring device generally include a thermal contact configured to contact a body of the subject; a heating element adjacent to the thermal contact and configured to heat the thermal contact; a thermal sensor configured to sense a temperature of the thermal contact and to obtain a plurality of body temperature measurements at a plurality of times; and a microcontroller configured to activate and deactivate the heating element, receive the plurality of measurements from the thermal sensor, determine parameters of a model used for predicting body temperature based on the plurality of measurements, predict a body temperature of the subject based on the model, and output the predicted body temperature as a current temperature reading.

In exemplary embodiments, the model includes:

t(time)=(Tₑ−T₀)(1−e^{-αt})+T₀,

where t=time, T₀=an initial temperature, Tₑ=a final temperature, and α=a thermal time constant, and determining the parameters of the model comprises determining values of α and Tₑ.

In several embodiments, the microcontroller is further configured to continue to obtain a plurality of body temperature measurement at a plurality of times after the predicted body temperature is outputted; and update the predicted body temperature based on the continued obtainment of the plurality of body temperature measurements at a plurality of times.

In one or more embodiments, the temperature monitoring device further includes an adhesive patch incorporating the heating element.

According to one or more embodiments, another method of measuring body temperature of a subject is provided. The method generally includes activating a heating element of a temperature monitor attached to a body of the subject for a predetermined amount of time; obtaining, by the temperature monitor, a plurality of body temperature measurements at a plurality of times for the subject for a time greater than the predetermined amount of time; determining, by the temperature monitor, parameters of a model used for predicting body temperature, based on the plurality of measurements; predicting, by the temperature monitor, a body temperature based on the model; and outputting, by the temperature monitor, the predicted body temperature as a current temperature reading.

In some embodiments, the model includes an exponential model for temperature, and wherein determining the parameters of the model includes determining values of a plurality of parameters in the exponential model. In certain embodiments, the model includes:

t(time)=(Tₑ−T₀)(1−e^{-αt})+T₀,

where t=time, T₀=an initial temperature, Tₑ=a final temperature, and α=a thermal time constant, and determining the parameters of the model includes determining values of α and Tₑ.

In several embodiments, predicting a body temperature of the subject based on the model includes determining a value of a of the model; and inputting a time into the model.

In various embodiments, the method further includes continuing to obtain a plurality of body temperature measurements at a plurality of times after the predicted body temperature is outputted; and updating the predicted body temperature based on the continued obtainment of the plurality of body temperature measurements at a plurality of times. In several embodiments, the plurality of times includes predetermined intervals.

In certain embodiments, the heating element is activated when the temperature monitor detects that the temperature monitor is attached to the body of the subject. In some embodiments, the heating element is embedded in an adhesive patch of the temperature monitor.

According to one or more embodiments, another temperature monitoring device is provided. The temperature monitoring device generally includes a thermal contact configured to contact a body of the subject; a heating element adjacent to the thermal contact and configured to heat the thermal contact; a thermal sensor configured to sense a temperature of the thermal contact and to obtain a plurality of body temperature measurements at a plurality of times for a time greater than a predetermined amount of time; and a microcontroller configured to activate the heating element for the predetermined amount of time, receive the plurality of measurements from the thermal sensor, determine parameters of a model used for predicting body temperature based on the plurality of measurements, predict a body temperature of the subject based on the model, and output the predicted body temperature as a current temperature reading.

In various embodiments, the model includes:

t(time)=(Tₑ−T₀)(1−e^{-αt})+T₀,

where t=time, T₀=an initial temperature, Tₑ=a final temperature, and α=a thermal time constant, and determining the parameters of the model includes determining values of α and Tₑ

In several embodiments, the microcontroller is further configured to continue to obtain a plurality of body temperature measurement at a plurality of times after the predicted body temperature is outputted; and update the predicted body temperature based on the continued obtainment of the plurality of body temperature measurements at a plurality of times.

In various embodiments, the temperature monitoring device further includes an adhesive patch incorporating the heating element.

Generally, any creation, storage, processing, and/or exchange of user data associated the method, apparatus, and/or system disclosed herein is configured to comply with a variety of privacy settings and security protocols and prevailing data regulations, consistent with treating confidentiality and integrity of user data as an important matter. For example, the apparatus and/or the system may include a module that implements information security controls to comply with a number of standards and/or other agreements. In some embodiments, the module receives a privacy setting selection from the user and implements controls to comply with the selected privacy setting. In other embodiments, the module identifies data that is considered sensitive, encrypts data according to any appropriate and well-known method in the art, replaces sensitive data with codes to pseudonymize the data, and otherwise ensures compliance with selected privacy settings and data security requirements and regulations.

Persons skilled in the art will recognize that the apparatus, systems, and methods described above can be modified in various ways. Accordingly, persons of ordinary skill in the art will appreciate that the embodiments encompassed by the present disclosure are not limited to the particular exemplary embodiments described above. In that regard, although illustrative embodiments have been shown and described, a wide range of modification, change, and substitution is contemplated in the foregoing disclosure. It is understood that such variations may be made to the foregoing without departing from the scope of the present disclosure. Accordingly, it is appropriate that the appended claims be construed broadly and in a manner consistent with the present disclosure.

What is claimed is:

1. A method for measuring body temperature of a subject, comprising:
obtaining, by a temperature monitor attached to a body of the subject, a plurality of body temperature measurements at a plurality of times for the subject;
determining, by the temperature monitor, parameters of a model used for predicting temperature, based on the plurality of measurements;
predicting, by the temperature monitor, a body temperature of the subject based on the model; and
outputting, by the temperature monitor, the predicted body temperature as a current temperature reading.

2. The method of claim 1, wherein the model comprises an exponential model for temperature, and wherein determining the parameters of the model comprises determining values of a plurality of parameters in the exponential model.

3. The method of claim 1, wherein the model comprises:
t(time)=(Tₑ−T₀)(1−e^{-αt})+T₀,
where t=time, T₀=an initial temperature, Tₑ=a final temperature, and α=a thermal time constant, and
wherein determining the parameters of the model comprises determining values of α and Tₑ.

4. The method of claim 3, wherein predicting a body temperature of the subject based on the model comprises:
determining a value of a of the model; and
inputting a time into the model.

5. The method of claim 1, further comprising:
continuing to obtain a plurality of body temperature measurements at a plurality of times after the predicted body temperature is outputted; and
updating the predicted body temperature based on the continued obtainment of the plurality of body temperature measurements at a plurality of times.

6. The method of claim 1, wherein the plurality of times comprises predetermined intervals.

7. The method of claim 1, further comprising activating a heating element coupled to the temperature monitor.

8. The method of claim 7, wherein the heating element is activated when the temperature monitor detects that the temperature monitor is attached to the body of the subject.

9. The method of claim 7, wherein activating the heating element comprises activating the heating element for a predetermined amount of time; and wherein obtaining the plurality of body temperature measurements comprises obtaining the plurality of body temperature measurements over a period of time that is greater than the predetermined amount of time.

10. A temperature monitoring device for measuring body temperature of a subject, which comprises:
a thermal sensor configured to sense a body temperature and to obtain a plurality of body temperature measurements at a plurality of times for the subject; and
a microcontroller configured to:
receive the plurality of measurements from the thermal sensor,
determine parameters of a model used for predicting body temperature based on the plurality of measurements,
predict a body temperature of the subject based on the model, and
output the predicted body temperature as a current temperature reading.

11. The temperature monitoring device of claim 10, wherein the model comprises:
t(time)=(Tₑ−T₀)(1−e^{-αt})+T₀,
where t=time, T₀=an initial temperature, Tₑ=a final temperature, and α=a thermal time constant, and
wherein determining the parameters of the model comprises determining values of α and Tₑ.

12. The temperature monitoring device of claim 10, wherein the microcontroller is further configured to:
continue to obtain a plurality of body temperature measurement at a plurality of times after the predicted body temperature is outputted; and
update the predicted body temperature based on the continued obtainment of the plurality of body temperature measurements at a plurality of times.

13. The temperature monitoring device of claim 10, further comprising a thermal contact configured to contact the body of the subject and a heating element adjacent to the thermal contact, wherein the microcontroller is configured to activate the heating element.

14. The temperature monitoring device of claim 13, wherein the microcontroller is further configured to:
detect that the temperature monitoring device is attached to the body of the subject, and
activate the heating element upon the detection.

15. The temperature monitoring device of claim 13, further comprising an adhesive patch incorporating the heating element.

16. A temperature monitoring device, which comprises:
a thermal contact configured to contact a body of the subject;
a heating element adjacent to the thermal contact and configured to heat the thermal contact;
a thermal sensor configured to sense a temperature of the thermal contact and to obtain a plurality of body temperature measurements at a plurality of times for a time greater than a predetermined amount of time; and
a microcontroller configured to:
activate the heating element for the predetermined amount of time,
receive the plurality of measurements from the thermal sensor,
determine parameters used for predicting body temperature based on the plurality of measurements,
predict a body temperature of the subject based on the parameters, and
output the predicted body temperature as a current temperature reading.

17. The temperature monitoring device of claim 16, wherein the parameters are part of a model, wherein the model comprises:
t(time)=(Tₑ−T₀)(1−e^{-αt})+T₀,
where t=time, T₀=an initial temperature, Tₑ=a final temperature, and α=a thermal time constant, and
wherein determining the parameters of the model comprises determining values of a and Tₑ.

18. The temperature monitoring device of claim 16, wherein the microcontroller is further configured to:
continue to obtain a plurality of body temperature measurement at a plurality of times after the predicted body temperature is outputted; and
update the predicted body temperature based on the continued obtainment of the plurality of body temperature measurements at a plurality of times.

19. The temperature monitoring device of claim 16, further comprising an adhesive patch incorporating the heating element.

20. The temperature monitoring device of claim 16, wherein the microcontroller is further configured to:
detect that the temperature monitoring device is attached to the body of the subject, and
activate the heating element upon the detection.
